(19) United States
(12) Plant Patent Application Publication
(10) Pub. No.: US 2004/0168240 P1
(43) Pub. Date: Aug. 26, 2004

(54) Artichoke plant named BCS 7-12

(76) Inventor: Medhat Chahbandar, Torreilles (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/371,880

(22) Filed: Feb. 20, 2003

(51) Int. Cl. A01H 5/00

(52) U.S. Cl. PLT/258

(57) ABSTRACT

A new and distinct cultivar of Artichoke plant named ‘BCS 7-12’. This new variety is characterized by red or violet bud color. Further, ‘BCA 7-12’ has a significantly larger fruit size and an earlier maturation compared to other red artichoke varieties. This variety has also shown to be more vigorous than its parents with a larger leaf area.

### BACKGROUND OF THE INVENTION

The present invention comprises a new and distinct cultivar of artichoke plant, botanically known as Cynara scolymus L., and herein referred to by the cultivar name ‘BCS 7-12’. This new variety is characterized by red or violet bud color, and a significantly larger size and an earlier maturation compared to other red artichoke varieties known to the inventor. This variety has also shown to be more vigorous than its parent with a very large leaf area.

Cynara scolymus L., commonly known as Globe artichoke, is a perennial herb and is a member of the family Asteraceae, also known as the Compositae family. Globe artichokes comprise: leaves, which are pinnately lobed, but primarily spineless; globose capitula composed of overlapping layers of large involucral bracts; and receptacles, which are enlarged and fleshy. Globe artichoke plants are essentially grown for the production of the immature flower heads (or buds), which are considered vegetable delicacies. Fresh artichokes may be steamed, boiled, or baked after which the fleshy receptacle, inner and outer bracts, and parts of the floral stem may be eaten.

The new cultivar is a product of a planned breeding program carried out by the inventor, M. Chahbandar near Perpignan, France. It was selected from a hybrid cross of the artichoke variety Chrysantheme and the artichoke variety BH 35-22. The inventor performed asexual reproduction of the new cultivar via division. It was demonstrated that the combination of characteristics as herein disclosed for the new cultivar are firmly fixed and retained through successive generations of asexual reproduction.

### BRIEF SUMMARY OF THE INVENTION

The following traits have been repeatedly observed and are determined to be basic characteristics of ‘BCS 7-12’. These traits in combination distinguish the artichoke variety as a new and distinct cultivar. ‘BCA 7-12’ is characterized by a red or violet fruit bud color. It has a significantly larger size (weight can be as much as 600-650 gm.) and an earlier maturation compared to other red artichoke varieties known to the inventor. ‘BCS 7-12’ has also proven to be more vigorous than its parents with a larger leaf area.

‘BCS 7-12’ has not been observed under all possible environmental conditions. The phenotype of the new cultivar may vary with variations in environment such as temperature, humidity, light intensity and day length, without any change in the genotype. However, the following observations, measurements and values, except for seed observations, describe the new cultivar as grown in Lompoc, Calif. under conditions which closely approximate those generally used in horticultural practice. Seed observations were carried out in Perpignan, France.

Lompoc is located on California's central coast in Santa Barbara County. Conditions can vary in the summer months. Air temperatures can range between the low 60's to 80's. The relative humidity is generally high. Prevailing winds are northwesterly and rainfall averages 12 inches per year.

In the following description, holding quality was measured by the physical appearance of the head. This includes the head's appearance following 3 and 7 day storage periods in cold storage at 34° F. The head's exterior (oxidation) was observed at each of the two observation points. Browning and blackening of plant tissue was evaluated as light, moderate and extreme. Overall storage response was measured by observing heads following 3 and 7 day cold storage periods. These observations concentrated on visible color variability and/or presence of lesions or other cosmetic anomalies. Leaf ratio (L/W) was determined by dividing representative leaf sample lengths by their width. Finally, head response (weather) was determined by observing the heads at maturity. These field observations focus on the presence or absence of bronzing, necrotic or chlorotic lesions or any abiotic responses to environmental conditions. AFLP (DNA analysis) was conducted on each parental selection.

### BRIEF DESCRIPTION OF THE DRAWINGS

This new variety is illustrated by the accompanying photographic drawings, with such colors in the photograph being as true to those of the plant as can be reasonably obtained from conventional photographic procedures. In such photographs:

FIG. 1 shows a side elevational view of ‘BCS 7-12’s fruit in full color.

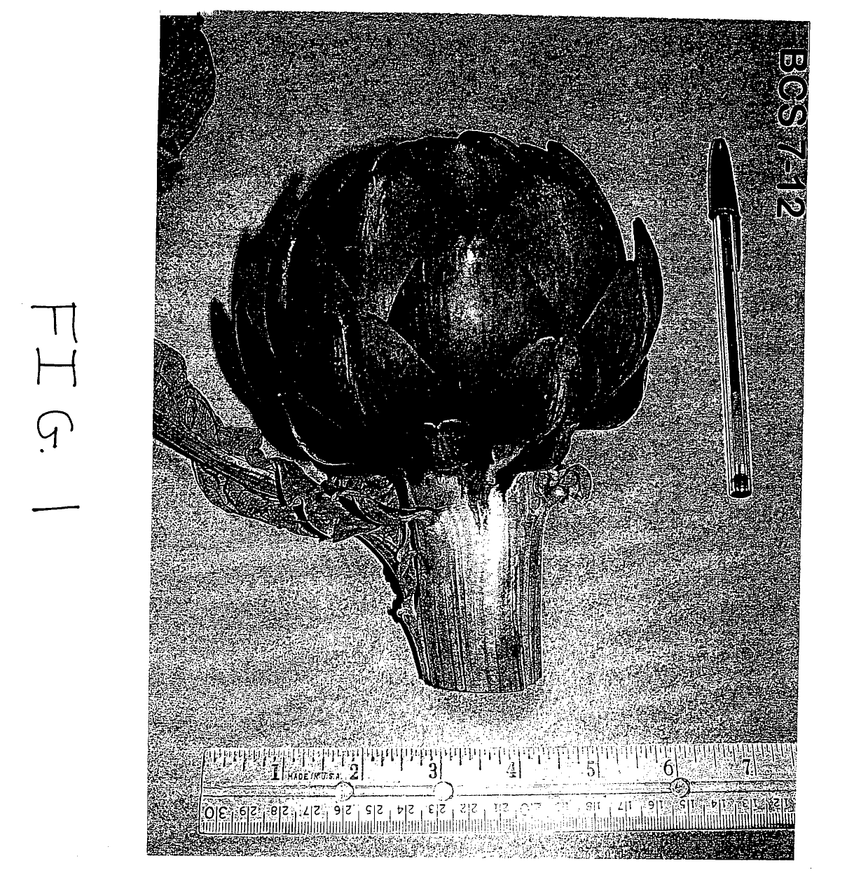

FIG. 2 shows a side elevational view of ‘BCS 7-12’s foliage in full color.

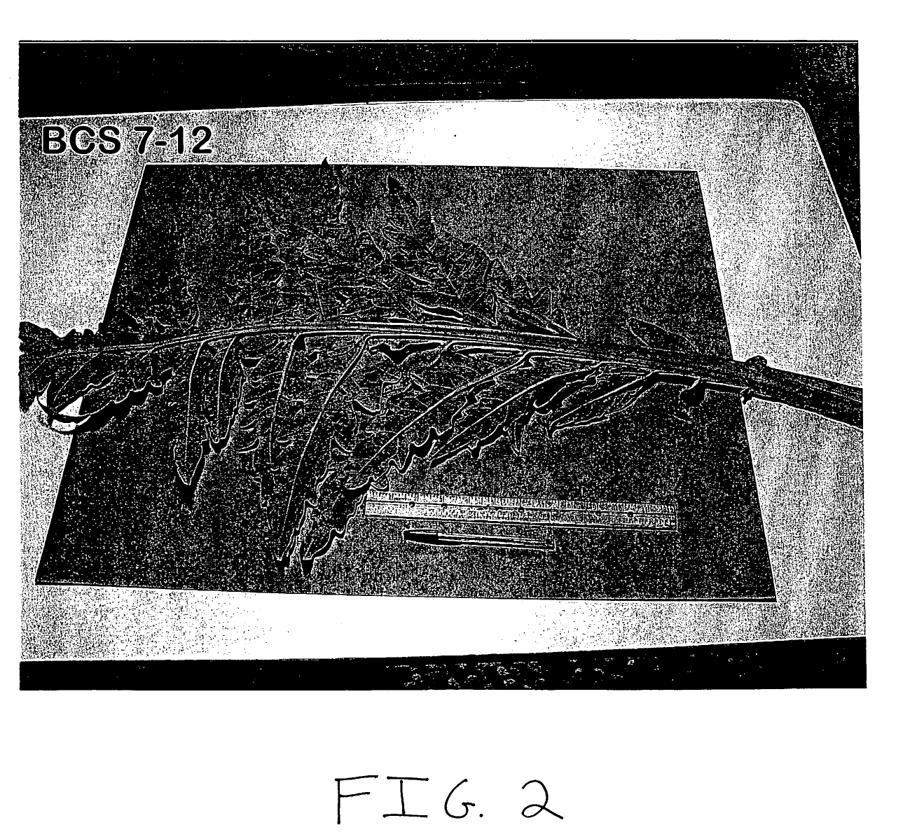

FIG. 3 shows a close-up view of the vertical cross-section of ‘BCS 7-12’s fruit in full color.

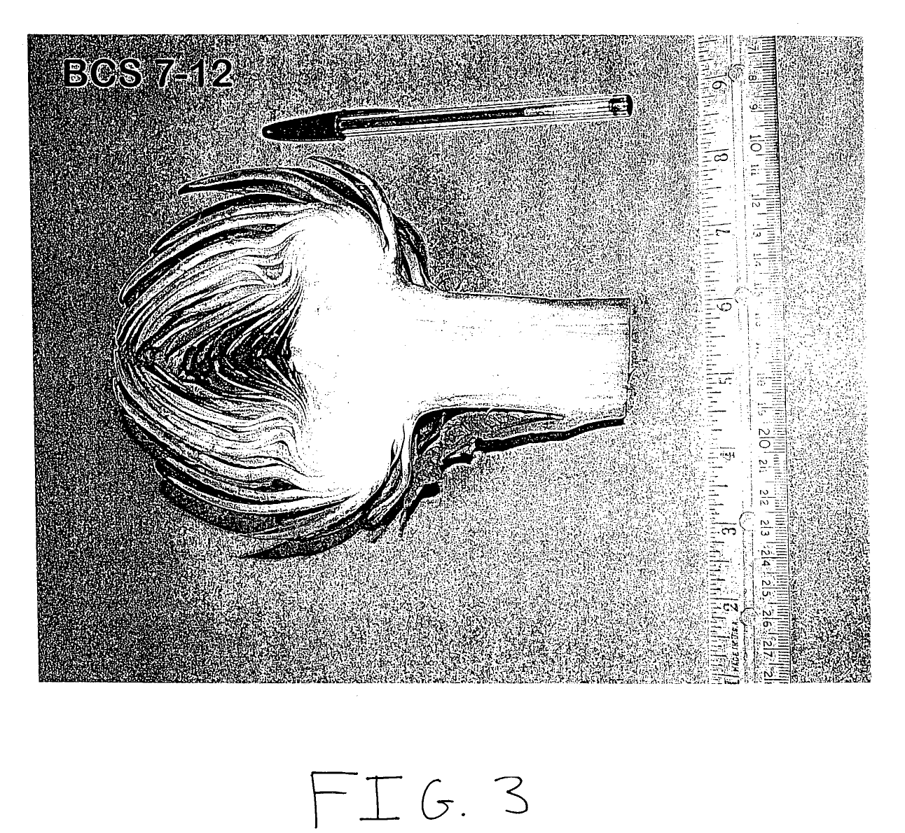

### DETAILED DESCRIPTION

The following observations, measurements, and values describe the new artichoke plant is based upon observations of plants grown in Lompoc, Calif. The data was collected from tissue culture plants grown in Perpignan and acclimatized in California. Selections were made in outdoor field settings. The plants were grown in the soil in rows where row spacing (bed centers) was at 80 inches, and individual plant spacing at 24 inches. The plants were then planted in July and the observations described herein taken in December of the next year. Color references are measured against The Royal Horticultural Society Colour.

General:

Parentage.—Hybrid cross between Chrysantheme and BH 35-22.

Classification.—Cynara Scolymus L. cv BCS 7-12.

Propagation.—Asexual production by division.

Plant:

Height.—83.9-111.8 cm; average 100.1 cm.

Width.—177.8-236.2 cm; average 205.2 cm.

Form.—Full.

Growth habit.—Upright.

Vigor.—Vigorous.

Side shoots:

Number.—7.2 per plant.

Development.—Vigorous.

Color.—Green group 143 b-c.

Foliage density.—Dense.

Capitulum:

Primary size.—Approximately 12.4 cm.

Shape.—Round compact with a slight pyramidal.

Texture.—Hard and smooth.

Fragrance.—Tangy with citrus overtones.

Bract:

Length.—Approximately 7 cm.

Width.—Approximately 4 cm.

Shape.—Ovate, medium width of the base, medium length, thick, and longer than broad.

Texture.—Hard and smooth.

Number.—Approximately 152 per head.

Color (inner).—Green group 138 a-b, and Grayed purple group 183 c-d at the tip.

Color (outer).—Grayed Purple group 183 a-b.

Firmness.—Firm and fleshy with thick basal thickness.

Spinosity.—None, or minimal — less than 1 mm.

Basal thickness.—6-17 mm.

Miscellaneous:

Heart description.—Slight concave full more flat than Green Globe, well developed with thick bract connection.

Heart color.—Yellow green group 145 d.

Receptacle thickness.—1.7 cm.

Florets.—Sterile.

Pappus length.—1.8 cm.

Pappus color.—Yellow green group 145 d.

Head firmness.—Firm, heads are dense and solid.

Gloss.—High; more glossy than Green Globe.

Cold storage response.—Some minor cut stem discoloration. After 7 days there is some fading of red/purple color.

Head response (weather).—After frosts the outer epidermal layer of the outer bract can separate, thus exposing a green layer underneath. Further, after prolonged exposure to sunlight the purple color becomes slightly greenish.

Foliage:

Shape.—Long strap-like leaves with regular dentate.

Length.—Approximately 116.8-138.4 cm.

Width.—Approximately 62.2-73.7 cm.

Main stem diameter.—7.0-9.2 average 8.0 cm.

Distance between main lobes.—Approximately 7.4-8.9 cm.

Leaf ratio.—Approximately 1.8-2.1 cm.

Leaf areas.—Approximately 3,038-4,014 cm sq.

Upper leaf surface color.—Green group 136 a-b.

Lower leaf surface color.—Green group 139 b-c.

Texture.—Slight but uniformly textured.

Pubescence.—Sparse, some visible pubescence on lower leaf surface.

Lobe width.—Approximately 11.4-14.9 cm.

Lobe indentation.—Approximately 7.0-7.6 cm.

Petiole width France.—Approximately 6.0 cm.

Petiole width.—Approximately 3.8-5.8 cm.

Petiole thickness.—Approximately 2.2-2.5 cm.

Petiole color.—Green group 139 c.

Seeds:

Shape.—Ovoid asymmetrical.

Number.—Very irregular.

Length.—8-10 mm.

Color.—White yellow.

What is claimed is:

1. The new distinct variety of artichoke plant substantially as shown and described, characterized particularly as to novelty by the characteristics listed above.
